(19) United States
(12) Patent Application Publication
(10) Pub. No.: US 2001/0012947 A1
(43) Pub. Date: Aug. 9, 2001

(54) Brush-type separating tool

(76) Inventors: Shuichi Ohashi, Hyogo (JP); Nobuhiro Kagaminuma, Fukushima (JP); Tetsuya Kiyono, Fukushima (JP); Miki Ishiguro, Osaka (JP)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/808,868

(22) Filed: Mar. 15, 2001

Related U.S. Application Data
(63) Continuation of application No. 09/254,611, filed on Mar. 10, 1999.

(30) Foreign Application Priority Data
Nov. 12, 1996 (JP) 314,141/1996

(51) Int. Cl. A61B 17/00

(52) U.S. Cl. 606/190; 606/84

(57) ABSTRACT

The invention provides a separating tool for undoing adhesion between a vessel, such as a blood vessel or cystic duct, and another tissue or peeling off a serous membrane.

A handle portion to be held by the surgeon, a head portion on which a plurality of filament bundles are arranged, and a connective portion for connecting the handle portion to the head portion are provided.

### TECHNICAL FIELD

The present invention relates to a separating tool for undoing adhesion between a vessel, such as a blood vessel or cystic duct, or peeling a serous membrane mainly in celiotomies or endoscopic surgeries.

### BACKGROUND ART

Basic surgical manipulations for performing an operation include incision, excision, disconnection, separation, ligature, suture, and hemostasis. Moreover, to smoothly perform these surgical manipulations, a specialized tool is used for each. Among the above manipulations, the separating which is the most basic manipulation of a surgical operation is carefully performed manually a pair of separating forceps or a scalpel so that blood vessel walls and internal organs are not damaged.

In general, in a surgery, mainly a celiotomy, to expose the portion to be operated upon by undoing adhesion between a vessel, an internal organ and some other tissue or peeling a serous membrane, separation is often accomplished by gently peeling the adhering tissue off by holding a compressed piece of gauze with a pair of forceps or the like and sliding it along the portion to be treated.

An endoscopic operation, in order first to approach the patient a portion in whose body cavity is to be treated, begins with piercing the patient's skin and body cavity with a trocar provided with a throughole and thereby securing a duct for inserting an endoscope and a treatment tool, and every subsequent surgical manipulation is performed through this trocar. Because every procedure of separation is done through the trocar, either a pair of separating forceps (dissector) for endoscopic surgery is used, or a compressed piece of gauze is held with a pair of nipping forceps for endoscopic surgery and slide along the portion to be treated.

A Kelly clamp used for celiotomy or separating forceps (so-called Maryland-type separating forceps whose tips are curved) used for endoscopic surgery makes it possible to gently peel off tissues. However, as shown in FIG. 1, the forceps of this type expand the portion to be separated by inserting their tips. Therefore, to strip and expose a fragile tissue with care a highly sophisticated skill is necessary because the sharp portion of the forceps may damage a blood vessel or internal organ.

Although the use of a compressed piece of gauge held between nipping forceps and inserted into the body cavity for a separating procedure as shown in FIG. 2 is tender to the blood vessel wall or tissue, swelling of the piece of gauze by being wet with blood or the like makes the gauze impossible to perform the separating function, and accordingly this entails the trouble of having to replace the piece of gauze frequently. While repeating this troublesome procedure, the piece of gauze, not held with a sufficient nipping force for the separating action, may often be inserted into the body cavity, and it is feared that the piece of gauze may be dropped within the body cavity when under endoscopic observation. Moreover, since general-purpose forceps do not embody consideration for durability against the holding of a piece of gauze, the service life of the forceps would be significantly reduced.

It is an object of the present invention to provide a separating tool for use in celiotomies and endoscopic surgeries, making possible very safe and easy separation irrespective of the experience of the surgeon by preventing a blood vessel or the like from being damaged by a sharp portion of forceps when separating the blood vessel from another tissue and moreover, preventing a piece of gauze from being dropped into the body cavity of the patient and eliminating the trouble of replacing the piece of gauze wet and swelled by blood.

### DISCLOSURE OF INVENTION

To solve the above problems, the present invention makes it possible for surgeon to safely and easily separate a blood vessel, cystic duct or the like by using a separating tool provided with a handle portion to be held by the surgeon and a head portion on which a plurality of filament bundles of appropriate hardness are arranged.

A cholecystectomy is described below as an example, which is a manipulation to extract the cholecyst from the body of a patient by celiotomy when the patient shows a symptom such as chronic cholecystitis involving calculuses or cholecystic polyps.

Separating actions until the extraction of the cholecyst include the following: first the cholecyst is exposed mainly by undoing the adhesion of greater and lesser omenta around the cholecyst; then a serous membrane is peeled from the region of the cholecyst to the common bile duct and developing the portion called the Calot triangle to fully expose the cystic duct and the cystic artery and accurately clip them off; and the portion called the cholecystic base where the cholecyst and the liver stick to each other is separated to take the cholecyst out of the abdominal cavity. A separating tool according to the invention can be used for all of the separating actions stated above.

By sliding the head portion of a brush-type separating tool of the present invention, on which filament bundles are arranged, it is possible to peel off greater and lesser omenta adhering to the cholecyst and the serous membrane surrounding the cystic duct and the cystic artery by tender rubbing, and therefore it is unnecessary to frequente replace treatment tools. Moreover, it is possible to enable the tool to function as a pair of exclusion forceps by using a protrusion at the tip of the head portion and thereby raising the internal organs around to confirm the separated state.

### BRIEF DESCRIPTION OF DRAWINGS

FIG. 1 is an illustration of the working state of a pair of separating forceps for use in endoscopic surgeries;

FIG. 2 is an illustration of a separating action with a piece of gauze held by a pair of nipping forceps for use in endoscopic surgeries;

FIG. 3 is an illustration showing a brush-type separating tool for use in celiotomies which is a first embodiment of the present invention;

FIG. 4 is an illustration showing a brush-type separating tool for use in endoscopic surgeries, which is a second embodiment of the present invention;

FIG. 5 is an illustration showing a brush-type separating tool for use in endoscopic surgeries, which is a fourth embodiment of the present invention;

FIG. 6 is an illustration showing a brush-type separating tool for use in endoscopic surgeries, which is a fifth embodiment of the present invention used for a surgical operation using an endoscope;

FIG. 7 is a longitudinal section of the brush unit portion of said separating tool; and

FIG. 8 is a longitudinal section of the handle portion of said separating tool.

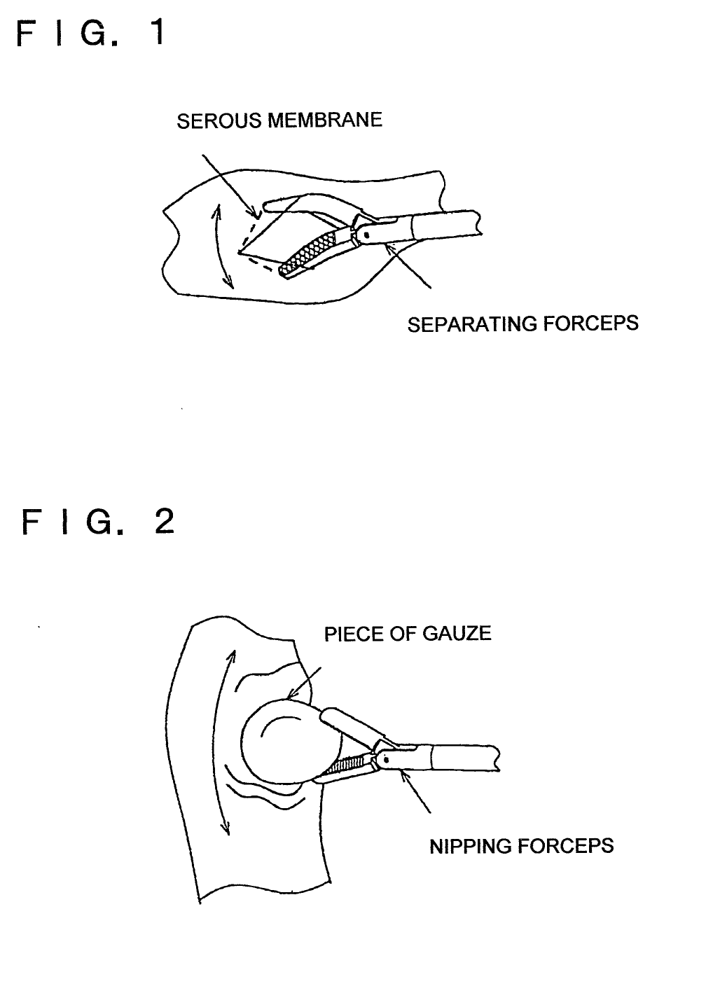

### BEST MODE FOR CARRYING OUT THE INVENTION

Embodiments of the present invention are described below in detail with reference to the accompanying drawings.

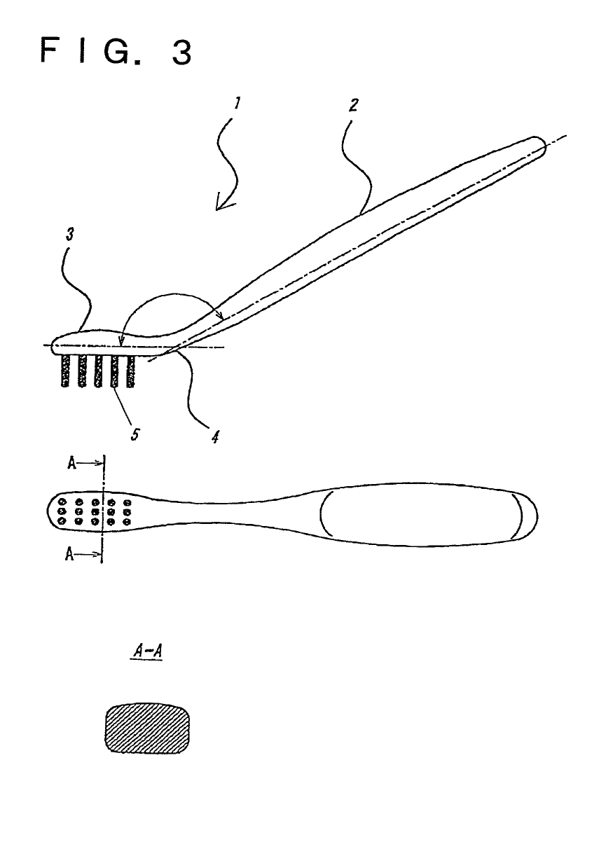

FIG. 3 is a side view of embodiment 1 of the present invention, showing the overall structure of a separating tool for use in celiotomies. As shown in FIG. 3, the separating tool 1 of this embodiment 1 comprises a slender rod-shaped handle portion 2, a head portion 3 having a flat bottom, and a connective portion 4 for connecting the root of the head portion 3 with the tip of the handle portion 2. The handle portion 2 forms an angle of 90° to 270° to the center axis of the head portion 3 at the tip of the center axis, but an angle of 120° to 150° is preferable for practical use. Filament planting holes with a diameter of 0.5 to 2.0 mm and a depth of 1.0 to 5.0 mm are formed at the bottom of the head portion 3 in 1 to 4 lines in the direction vertical to the center axis of the head portion 3 and in 2 to 8 rows in the direction horizontal to the center axis of same, and a plurality of filament bundles each of which is formed by sheaving together filaments with an individual diameter of 0.05 to 0.40 mm are secured in the filament planting holes so that they vertically extend from the bottom of the head portion in a length of 2 to 10 mm. The handle portion 2, connective portion 4, and head portion 3 are formed by integral molding or cutting of an inexpensive synthetic resin or the like.

Thus, embodiment 1 makes it possible to safely and easily separate a vessel or the like in a celiotomy because the surgeon can hold the handle portion 2 in his wrist at a natural angle when sliding the head portion 3 as the head portion 3 is at an inclination vis-a-vis the head portion based on human engineering wisdom. Moreover, by building a motor in the handle portion 2 to electrically slide the head portion 3, it is possible to reduce the load on the surgeon. Furthermore, by forming the head portion 3 and the handle portion 2 of an inexpensive synthetic resin, a separation tool of a so-called disposable type to be discarded after use is realized, a feature effective for the prevention of nosocomial infection.

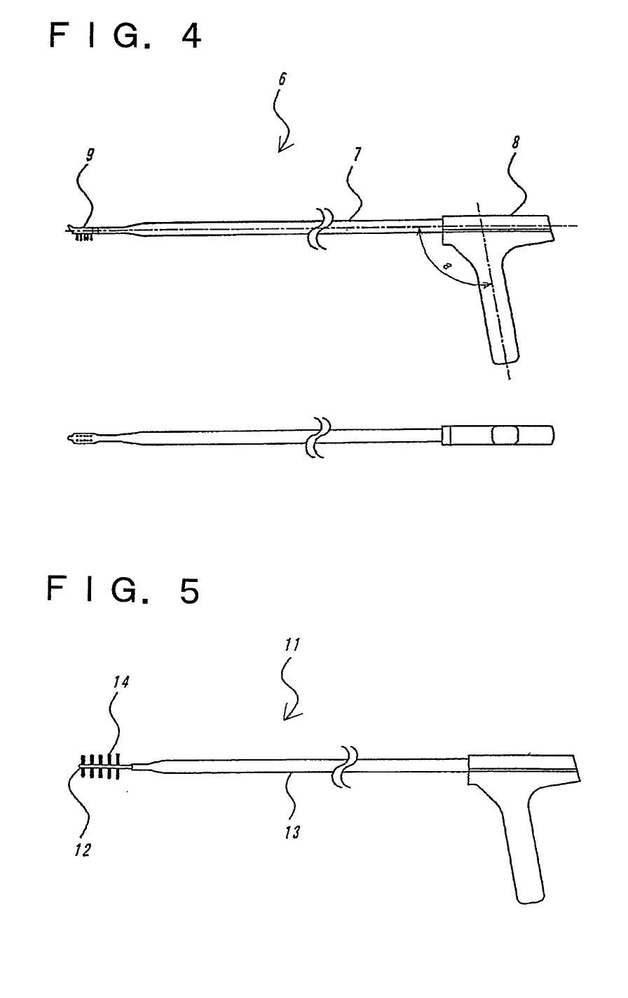

FIG. 4 is a side view of embodiment 2 of the present invention, showing the overall structure of a separating tool for use in endoscopic surgeries. In the separating tool 6 of embodiment 2, a handle portion 8 is secured to the root of a connective portion 7 made of a long extension member with a length of 300 to 500 mm and a diameter of 8 to 11 mm forming an angle of 90° to 270° to the center axis of the connective portion 7 at the tip of the center axis as shown in FIG. 4. It is preferable to set the angle to 210° to 240° for practical use. Moreover, similarly to embodiment 1, the handle portion 8, connective portion 7, and head portion 9 are formed by integral molding or cutting of an inexpensive synthetic resin. After use, embodiment 2 can be treated as a disposable-type separating tool.

Thus, embodiment 2, provided with the long connective portion 7, makes it possible to manipulate the head portion 9 inserted into the body of a patient through a trocar and safely and easily peel off a vessel or the like with the appropriate hardness and friction of the filaments. Moreover, because the tip of the head portion 9 is formed in a spatular protrusion, it also functions as a pair of exclusion forceps for confirming the separated state by raising the internal organs around with the protrusion.

In embodiment 3, the handle portion 8 and the connective portion 7 of embodiment 2 are formed of a material consisting of a metal such as stainless steel or a titanium alloy. When forming a separating tool as mentioned above, only the head portion is made of an inexpensive synthetic resin and the root of the head portion 9 is removably fitted to the tip of the connective portion 7. Therefore, only the head portion 9 is disposable after use and the holding and connective portions can be reused many times if sterilized; therefore, this also contributes to reducing the cost of surgery.

A separating tool 11, which is embodiment 4 of the present invention, shown in FIG. 5 is constituted by fitting a head portion 12 provided with a plurality of filament bundles each of which is formed by sheaving fine filaments together, that is, having a shape like a so-called inter-dental brush to the tip of a connective portion 13. Thus, embodiment 4 makes it possible to perform a separating action even in a narrow gap by inserting its head portion 12 when a vessel or serous membrane is to be peeled off.

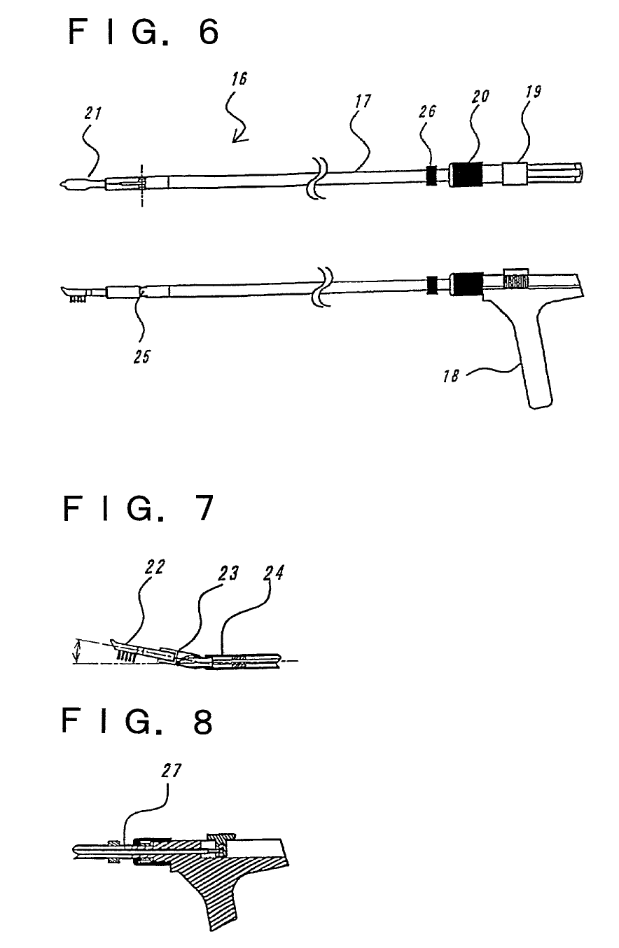

FIGS. 6 to 8 are side views of embodiment 5 of the present invention, showing the overall structure of a separating tool for use in endoscopic surgeries. A separating tool 16 of embodiment 5 comprises a handle 18, a connective portion 17 made of an extension tube, a support tube 24, and a brush unit 21. A guide groove extending in the direction horizontal to the axial direction is formed on both sides of the handle portion 18, and a slider 19 engaging with the guide groove is fitted to the handle portion 18 so as to be movable back and forth. Further, the connective portion 17 made of a sleeve-shaped extension tube is rotatably fitted to the tip of the handle portion 18 through a box-nut-shaped joint member 20.

A brush unit 21 comprises a brush head portion 22 and a housing 23, and the brush head portion 22 is detachably fitted to the tip of the housing 23.

The root of the housing 23 is connected to the tip of the support tube 24 through a connecting pin 25 so as to be rotatable around the connecting pin 25 in a range between −45° and +45° to the center axis of the connective portion 17. The root of the support tube 24 caps the tip of the connective portion 17. A long shaft 27 of which one end is spherical and the other end is provided with a cam follower is inserted into the connective portion 17 and the handle portion 18, a recess formed on the slider 19 engages with the sphere at one end of the shaft, and a cam groove formed on the housing 23 engages with the cam follower at the other end of the shaft 27. This arrangement makes it possible to pivot the brush unit 21 around the connecting pin 25 in a range of −45° to +45° to the center axis of the connective portion 17 by moving the slider 19 back and forth, causing the shaft 27 positioned within to move in response and thereby causing the cam follower at the tip of the shaft to move along the cam groove disposed in the housing 23. Further, by turning a rotary dial 26 fitted to the connective portion 17, it is possible to rotate the connective portion 17 and the brush unit 21 around the center axis of the connective portion 17. Therefore, it is possible to peel off a tissue even in a position which is in a dead angle to the trocar inserting direction.

Thus, embodiment 5 makes it possible to perform a manipulation even in a position where it is difficult to perform endoscopic separation by sliding the brush unit 21 and rotating the connective portion 17.

The following are the results of using a brush-type separating tool of the present invention for actual surgical operations to measure the effect of the separating tool.

(1) Blood vessel separation in gastrectomy (Two cases)

As the result of separating two out of four main blood vessels connected to the stomach with “filament bundles”, the vessels could be treated in an almost bloodless process.

(2) Blood vessel treatment in sigmoidectomy (Two cases)

As the result of separating the sigmoid artery and the tissue around an inferior mesenteric artery at the root of the sigmoid artery, it was possible to safely and bloodlessly perform a perfect separation as would be depicted an anatomical chart.

(3) Separation of cystic ducts and cystic arteries (Twenty cases)

Tissues around cystic ducts and around cystic arteries were separated in endoscopic cholecystectomies using a brush-type separating tool of the present invention and Maryland-type separating forceps in ten cases each. Table 1 shows the result of measuring the time required for separation.

Measurements

| | | | | | | | | | | | Average value |
|---|---|---|---|---|---|---|---|---|---|---|---|
| | 1 | 2 | 3 | 4 | 5 | 6 | 7 | 8 | 9 | 10 | (min.) |
| Brush-type | 7 | 5 | 8 | 13 | 4 | 6 | 10 | 7 | 5 | 6 | 7.1 |
| separating tool | | | | | | | | | | | |
| Maryland-type | 18 | 14 | 30 | 30 | 23 | 36 | 21 | 16 | 12 | 28 | 22.8 |
| separating forceps | | | | | | | | | | | |

As demonstrated by the comparison of the brush-type separating tool of the present invention with the commercial Maryland-type separating forceps, it is confirmed that the separating tool of the present invention makes it possible to very easily and safely separate cystic ducts and cystic arteries in a time approx. ⅓ of the time taken by the Maryland-type separating forceps.

As described above, a separating tool of the present invention makes it possible to very safely and easily perform separation without relying on any highly sophisticated skill when separating a vessel such as a blood vessel or a cystic duct in celiotomies or endoscopic surgeries and moreover, greatly shorten the time taken for the surgical operation.

1. A separating tool for use in surgical operations, comprising a handle portion to be held by the surgeon, a head portion on which a plurality of filament bundles are arranged, and a connective portion for connecting said handle portion to said head portion.

2. A separating tool for use in surgical operations according to claim 1, wherein the sectional form of a head portion perpendicular to its center axis is any one of a circle, ellipse, quadrangle, and triangle and a plurality of filament bundles each of which is obtained by sheaving together 8 to 12 filaments with an individual diameter of 0.05 to 0.40 mm are arranged on at least a part of the outer periphery of said head portion so that they extend in a length of 2 to 10 mm in the direction substantially vertical to the surface of said head portion.

3. A separating tool for use in surgical operations according to claim 1, wherein a handle portion is secured in a tilt angle of 90° to 270° to the center axis of the head portion at the tip of the center axis.
